(19) United States
(12) Patent Application Publication
(10) Pub. No.: US 2025/0319284 A1
(43) Pub. Date: Oct. 16, 2025

(54) INTRODUCER SHEATH WITH BLOOD BYPASS MECHANISM

(71) Applicant: BOSTON SCIENTIFIC SCIMED, INC., Maple Grove, MN (US)

(72) Inventors: Zachary Kumor, Plymouth, MI (US); Benjamin Jerome Bidne, Loretto, MN (US); Jeffrey Schneider, Dayton, MN (US); Lauren Ann Sandy, Minneapolis, MN (US); Daniel Presteng, Corcoran, MN (US)

(73) Assignee: BOSTON SCIENTIFIC SCIMED, INC., Maple Grove, MN (US)

(21) Appl. No.: 19/171,707

(22) Filed: Apr. 7, 2025

Related U.S. Application Data
(60) Provisional application No. 63/632,082, filed on Apr. 10, 2024.

(51) Int. Cl. A61M 25/06; A61M 25/00

(52) U.S. Cl. CPC A61M 25/0662; A61M 25/0043; A61M 25/0097; A61M 2025/0006; A61M 2205/0266; A61M 2205/7545

(57) ABSTRACT

A hub and sheath assembly configured to selectively fluidly couple a lumen of the sheath with an exterior thereof. The hub and sheath assembly may comprise a valve hub including a lumen extending therethrough. An elongate shaft may extend from a proximal end region to a distal end region and include a lumen extending therebetween. The elongate shaft may be coupled to the valve hub. A fluid bypass mechanism may extend through a sidewall of the elongate shaft. The fluid bypass mechanism may be configured to selectively fluidly couple the lumen of the elongate shaft with an exterior thereof.

### CROSS REFERENCE TO RELATED APPLICATIONS

The present application claims the benefit of U.S. Provisional Patent Application Ser. No. 63/632,082, filed Apr. 10, 2024, the disclosure of which is incorporated herein by reference.

### TECHNICAL FIELD

The present disclosure pertains to sheaths for removing and/or delivering intravascular medical devices. More specifically, the present disclosure relates to a sheath which may be disposed over an in-situ medical device.

### BACKGROUND

In various procedures for delivering intravascular medical devices, a sheath is inserted into a blood vessel of a patient, for example a femoral artery, and one or more medical devices may be advanced through the sheath and into the patient's vasculature. In various instances, the medical devices include catheters or other devices, such as a blood pump. The sheath may remain in place while the medical device is within the body to facilitate removal of the medical device when the procedure is complete. A device that is in use for an extended period of time (e.g., multiple days) may require minimum disruption to blood flow to avoid ischemia. In some cases, a sheath may reduce blood flow while it remains in the body. Thus, there is a need for improved sheaths for introduction and/or removal of medical devices that minimally disrupt blood flow while the medical device is in use.

### BRIEF SUMMARY

This disclosure provides design, material, manufacturing method, and use alternatives for medical devices, including introducer and/or removal sheaths.

In a first example, a hub and sheath assembly may comprise a valve hub including a lumen extending therethrough, an elongate shaft extending from a proximal end region to a distal end region and including a lumen extending therebetween, the elongate shaft coupled to the valve hub and a fluid bypass mechanism extending through a sidewall of the elongate shaft. The fluid bypass mechanism may be configured to selectively fluidly couple the lumen of the elongate shaft with an exterior thereof.

Alternatively or additionally to any of the examples above, in another example, the fluid bypass mechanism may comprise a movable flap formed in the sidewall of the elongate shaft.

Alternatively or additionally to any of the examples above, in another example, the movable flap may be configured to be deflected radially inwards.

Alternatively or additionally to any of the examples above, in another example, the hub and sheath assembly may further comprise a pull wire coupled to the fluid bypass mechanism.

Alternatively or additionally to any of the examples above, in another example, the pull wire may extend from the fluid bypass mechanism to the valve hub.

Alternatively or additionally to any of the examples above, in another example, the hub and sheath assembly may further comprise an actuation mechanism coupled to a proximal end of the pull wire.

Alternatively or additionally to any of the examples above, in another example, actuation of the actuation mechanism may be configured to move the fluid bypass mechanism between a generally closed configuration and an open configuration.

Alternatively or additionally to any of the examples above, in another example, the hub and sheath assembly may further comprise a mesh positioned over the elongate shaft adjacent to the fluid bypass mechanism.

Alternatively or additionally to any of the examples above, in another example, the hub and sheath assembly may further comprise a biasing mechanism extending between the elongate shaft and at least one side of the flap.

Alternatively or additionally to any of the examples above, in another example, the biasing mechanism may be configured to bias the flap into a radially inwards open configuration.

Alternatively or additionally to any of the examples above, in another example, the biasing mechanism may comprise a shape memory material.

Alternatively or additionally to any of the examples above, in another example, the fluid bypass mechanism may comprise a plurality of apertures extending through the sidewall of the elongate shaft.

Alternatively or additionally to any of the examples above, in another example, the hub and sheath assembly may further comprise an outer tubular member movably disposed over the elongate shaft.

Alternatively or additionally to any of the examples above, in another example, rotational and/or longitudinal actuation of the outer tubular member may be configured to selectively expose the fluid bypass mechanism.

Alternatively or additionally to any of the examples above, in another example, the fluid bypass mechanism may be positioned in the range of about 3 to 10 centimeters distal to the valve hub.

In another example, a hub and sheath assembly may comprise a valve hub including a lumen extending therethrough, an elongate shaft extending from a proximal end region to a distal end region and including a lumen extending therebetween, the elongate shaft coupled to the valve hub, and a fluid bypass mechanism extending through a sidewall of the elongate shaft. The fluid bypass mechanism may be configured to selectively fluidly couple the lumen of the elongate shaft with an exterior thereof.

Alternatively or additionally to any of the examples above, in another example, the fluid bypass mechanism may comprise a movable flap formed in the sidewall of the elongate shaft.

Alternatively or additionally to any of the examples above, in another example, the movable flap may be configured to be deflected radially inwards.

Alternatively or additionally to any of the examples above, in another example, the hub and sheath assembly may further comprise a pull wire coupled to the fluid bypass mechanism.

Alternatively or additionally to any of the examples above, in another example, the pull wire may extend from the fluid bypass mechanism to the valve hub.

Alternatively or additionally to any of the examples above, in another example, the hub and sheath assembly may further comprise an actuation mechanism coupled to a proximal end of the pull wire.

Alternatively or additionally to any of the examples above, in another example, actuation of the actuation mechanism may be configured to move the fluid bypass mechanism between a generally closed configuration and an open configuration.

Alternatively or additionally to any of the examples above, in another example, the hub and sheath assembly may further comprise a mesh positioned over the elongate shaft adjacent to the fluid bypass mechanism.

In another example, a hub and sheath assembly may comprise a valve hub including a lumen extending therethrough, an elongate shaft extending from a proximal end region to a distal end region and including a lumen extending therebetween, the elongate shaft coupled to the valve hub, and a fluid bypass mechanism extending through a sidewall of the elongate shaft, the fluid bypass mechanism configured to move between a first closed configuration and a second open configuration. The fluid bypass mechanism may be configured to selectively fluidly couple the lumen of the elongate shaft with an exterior thereof.

Alternatively or additionally to any of the examples above, in another example, the fluid bypass mechanism may be biased to the open configuration.

Alternatively or additionally to any of the examples above, in another example, the hub and sheath assembly may further comprise a biasing mechanism extending between the elongate shaft and at least one side of the flap.

Alternatively or additionally to any of the examples above, in another example, the biasing mechanism may be configured to bias the flap into a radially inwards open configuration.

Alternatively or additionally to any of the examples above, in another example, the biasing mechanism may comprise a shape memory material.

Alternatively or additionally to any of the examples above, in another example, the biasing mechanism may comprise a scaffold structure.

In another example, a hub and sheath assembly may comprise a valve hub including a lumen extending therethrough, an elongate shaft extending from a proximal end region to a distal end region and including a lumen extending therebetween, the elongate shaft coupled to the valve hub, and a fluid bypass mechanism extending through a sidewall of the elongate shaft. The fluid bypass mechanism may comprise a plurality of apertures extending through the sidewall of the elongate shaft.

Alternatively or additionally to any of the examples above, in another example, the hub and sheath assembly may further comprise an outer tubular member movably disposed over the elongate shaft.

Alternatively or additionally to any of the examples above, in another example, rotational and/or longitudinal actuation of the outer tubular member may be configured to selectively expose the fluid bypass mechanism.

Alternatively or additionally to any of the examples above, in another example, the plurality of apertures may be arranged in one or more circumferential arrays.

Alternatively or additionally to any of the examples above, in another example, the outer tubular member may comprise a radially inwardly extending protrusion, the radially inwardly extending protrusion may be configured to engage a mating slot formed in an outer surface of the elongate member.

Alternatively or additionally to any of the examples above, in another example, the fluid bypass mechanism may be positioned in the range of about 3 to 10 centimeters distal to the valve hub.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a side view of an introducer sheath extending into a blood vessel;

FIG. 2 is a cross-sectional view of a portion of the introducer sheath of FIG. 1 inserted into a blood vessel, and a medical device inserted into the introducer sheath;

FIG. 3A is a partial cross-sectional view of an illustrative introducer sheath extending into a blood vessel in a first configuration;

FIG. 3B is a partial cross-sectional view of the illustrative introducer sheath of FIG. 3A extending into a blood vessel in a second configuration;

FIG. 4 is a partial bottom view of the introducer sheath of FIG. 3B adjacent to the fluid bypass mechanism;

FIG. 5 is a partial bottom view of the introducer sheath of FIG. 3B adjacent to the fluid bypass mechanism having a mesh or screen;

FIG. 6A is a partial cross-sectional view of another illustrative introducer sheath disposed within a vessel and in a first, closed, configuration;

FIG. 6B is a partial cross-sectional view of the illustrative introducer sheath of FIG. 6A disposed within a vessel and in a second, open, configuration;

FIG. 7 is a partial bottom view of the introducer sheath of FIG. 6B adjacent to the fluid bypass mechanism;

FIG. 8A is a partial cross-sectional view of another illustrative introducer sheath in a first configuration; and

FIG. 8B is a partial cross-sectional view of the illustrative introducer sheath of FIG. 8A in a second configuration.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

Relative terms such as “proximal”, “distal”, “advance”, “retract”, variants thereof, and the like, may be generally be considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user.

The following detailed description should be read with reference to the drawings. The drawings, which are not necessarily to scale, depict illustrative embodiments and are not intended to limit the scope of the disclosure. Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

Some medical devices may extend percutaneously into the body and remain in position for an extended period of time (e.g., hours, days, weeks, etc.). An introducer sheath may be used to facilitate introduction of the medical device into the vasculature. For example, a sheath may be introduced into the femoral artery and one or more medical devices may be advanced through the sheath and into the patient's vasculature. In some embodiments, the medical device may include catheters or other devices, such as, but not limited to, a blood pump. The introducer sheath may remain within the body while the medical device remains in use with a hemostasis valve hub assembly helping to facilitate insertion of the medical device as well as helping to prevent blood from leaking during the medical procedure. When the medical procedure is complete, or the medical device is no longer needed, the introducer sheath may facilitate removal of the medical device. It is desirable for a medical device that is in use for a prolonged period of time (e.g., more than twenty-four hours) to minimally disrupt blood flow to avoid critical limb ischemia. However, an introducer sheath may reduce blood flow for as long as it remains within the body increasing the risk of ischemia. Some embodiments of the present disclosure are directed toward a sheath, such as, but not limited to, an introducer sheath, that can improve blood flow when the sheath remains in the body.

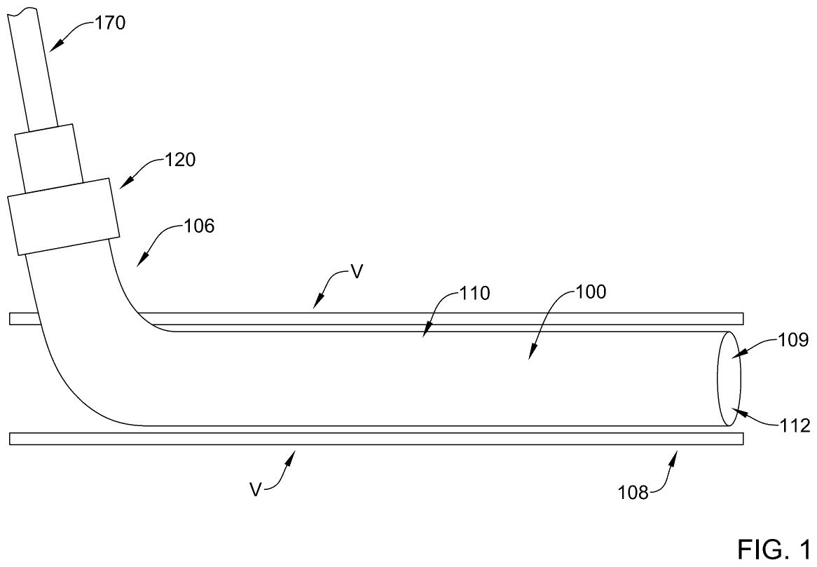

FIG. 1 illustrates a side view of an introducer sheath 100 inserted at least partially into a blood vessel V, shown in partial cross-section. In some embodiments, the introducer sheath 100 may be used for facilitating the passage of various medical devices, such as a catheter or a blood pump, as will be described further herein, through the introducer sheath 100 and into the blood vessel V. The introducer sheath 100 includes a proximal end region 106 proximate a proximal end of the introducer sheath 100 and a distal end region 108 proximate a distal end of the introducer sheath 100 that is opposite the proximal end region 106. An elongate shaft 110 of the introducer sheath 100 extends between the proximal end region 106 and the distal end region 108, and the elongate shaft 110 defines a lumen 112 of the introducer sheath 100. The introducer sheath 100 includes a proximal opening (not shown) adjacent the proximal end region 106 and a distal opening 109 adjacent to the distal end region 108, with the lumen 112 extending from the proximal opening to the distal opening 109. The introducer sheath 100, or components thereof, may be formed by various materials, such as polymeric and/or metallic materials. In some instances, the introducer sheath 100, such as the elongate shaft of the introducer sheath 100, may include an additional surface coating, such as but not limited to, silicone, polyethylene terephthalate (PET), or other applicable polymer.

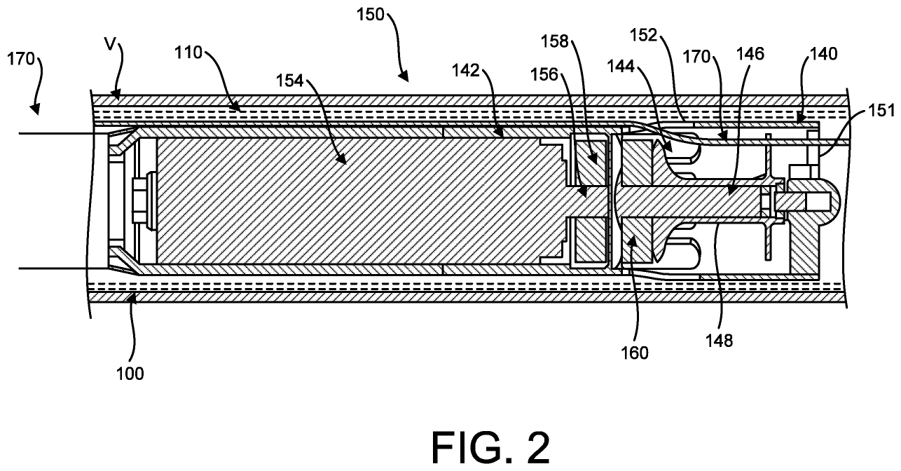

A hemostasis valve hub 120 (hereinafter “hub 120” for brevity) may be provided at the proximal end region 106 to provide access to the lumen 112 of the introducer sheath 100. The hub 120 may be configured for hemostasis by, for example, helping to prevent blood from leaking out of the introducer sheath 100 during use. For example, a medical device 170, such as a catheter or blood pump, may be inserted through the hub 120 and lumen 112 of the introducer sheath 100 and into the blood vessel V, and the hub 120 may maintain hemostasis between the medical device 170, the introducer sheath 100, and the external surroundings. In some embodiments, the medical device 170, may include and/or be coupled to a blood pump 150, shown in FIG. 2.

FIG. 2 illustrates a cross-sectional view of the elongate shaft 110 of the introducer sheath 100 of FIG. 1 upon insertion of a medical device, illustratively a blood pump 150, into the introducer sheath 100. As noted above, the medical device 170 of FIG. 1 may be coupled to or include the blood pump 150, with the medical device 170 extending outside the blood vessel V and the introducer sheath 100. The blood pump 150 may be advanced through the blood vessel V and positioned in a target location, such as a target cardiac location (e.g., the left ventricle), via the introducer sheath 100. The blood pump 150 may generally include an impeller assembly housing 140 and a motor housing 142. In some embodiments, the impeller assembly housing 140 and the motor housing 142 may be integrally or monolithically constructed (e.g., formed as single unitary structure). In other instances, the impeller assembly housing 140 and the motor housing 142 may be separate components. The impeller assembly housing 140 carries an impeller assembly 144 therein. The impeller assembly 144 may include an impeller shaft 146 and an impeller 148 that rotate relative to the impeller assembly housing 140 to drive blood through the blood pump 150. More specifically, the rotation of the impeller 148 causes blood to flow from a blood inlet 151 formed on the impeller assembly housing 140, through the impeller assembly housing 140, and out of a blood outlet 152 formed on the impeller assembly housing 140. In some embodiments, the impeller shaft 146 and the impeller 148 may be integrally formed as a single unitary structure, whereas, in other embodiments the impeller shaft 146 and the impeller 148 may be separate components. As shown in FIG. 2, the inlet 151 may be formed on an end portion of the impeller assembly housing 140 and the outlet 152 may be formed on a side portion of the impeller assembly housing 140. In other embodiments, the inlet 151 and/or the outlet 152 may be formed on other portions of the impeller assembly housing 140. In some embodiments, the impeller assembly housing 140 may be coupled to a distally extending cannula, and the cannula may receive and deliver blood to the inlet 151.

With continued reference to FIG. 2, the motor housing 142 carries a motor 154, and the motor 154 is configured to rotatably drive the impeller 148 relative to the impeller assembly housing 140. In the illustrated embodiment, the motor 154 rotates a drive shaft 156, which is coupled to a driving magnet 158. Rotation of the driving magnet 158 causes rotation of a driven magnet 160, which is connected to the impeller assembly 144. More specifically, in embodiments incorporating the impeller shaft 146, the impeller shaft 146 and the impeller 148 are configured to rotate with the driven magnet 160. In other embodiments, the motor 154 may be coupled to the impeller assembly 144 via other components. While the introducer sheath 100 is illustrated above with the use of the blood pump 150, various other medical devices may be used in conjunction with the introducer sheath 100 and the hemostasis valve hub 120.

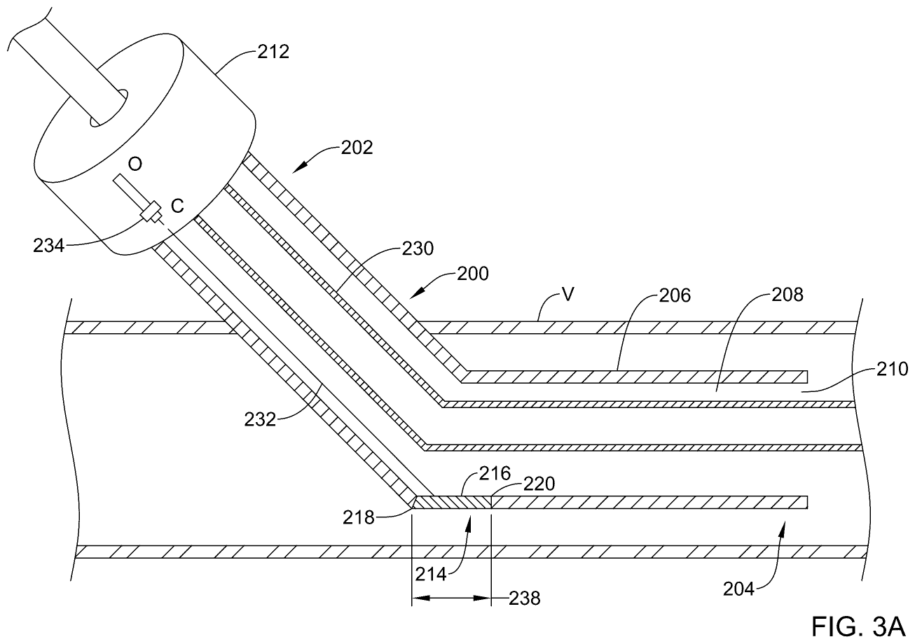

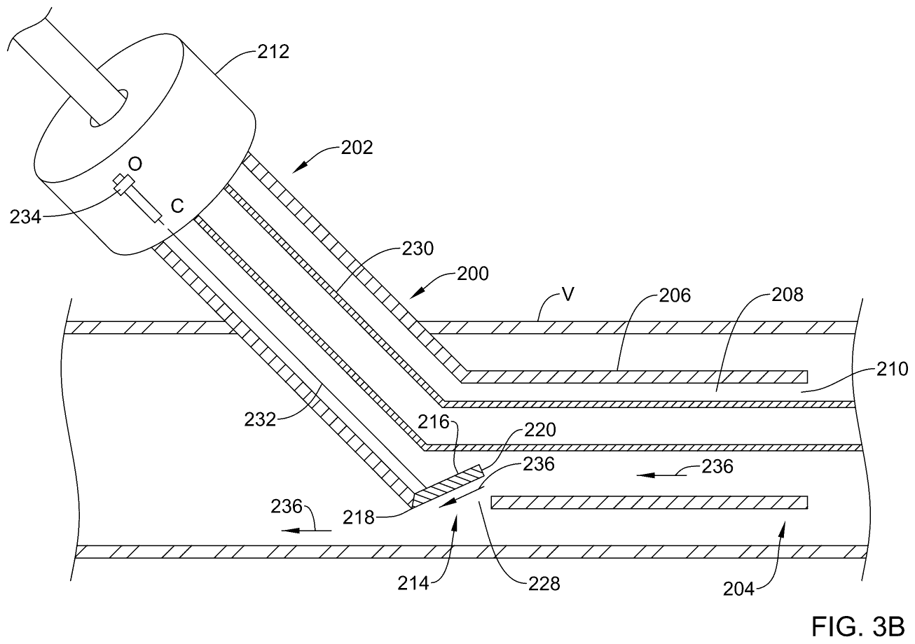

Some large bore access devices may have larger distal capsule sections which require introducer sheaths to have diameters or cross-sectional dimensions large enough to accommodate the cross-sectional dimension of the distal capsule section. However, larger diameter introducer sheaths may occlude or otherwise inhibit the flow of blood through the vessel which may lead to critical limb ischemia. It is contemplated that the introducer sheath 100 may include a blood bypass therein to improve blood flow proximal to the introduction point. FIG. 3A is a partial cross-sectional view of an illustrative introducer sheath 200 extending into a blood vessel V in a first configuration and FIG. 3B is a partial cross-sectional view of the illustrative introducer sheath 200 extending into a blood vessel V in a second configuration.

The introducer sheath 200 includes a proximal end region 202 proximate a proximal end of the introducer sheath 200 and a distal end region 204 proximate a distal end of the introducer sheath 200 that is opposite the proximal end region 202. While the illustrated embodiment shows the distal end region 204 of the introducer sheath 200 proximal to a distal end region (not explicitly shown) of the medical device 230, the distal end region 204 of the introducer sheath 200 may extend distal to a distal end region of the medical device 230 or may be adjacent to or near the distal end region of the medical device 230. An elongate shaft 206 of the introducer sheath 200 extends between the proximal end region 202 and the distal end region 204, and the elongate shaft 206 defines a lumen 208 of the introducer sheath 200. The introducer sheath 200 includes a proximal opening (not shown) adjacent the proximal end region 202 and a distal opening 210 adjacent the distal end region 204, with the lumen 208 extending from the proximal opening to the distal opening 210. The introducer sheath 200, or components thereof, may be formed by various materials, such as polymeric and/or metallic materials. In some instances, the introducer sheath 200, such as the elongate shaft of the introducer sheath 200, may include an additional surface coating, such as but not limited to, silicone, polyethylene terephthalate (PET), or other applicable polymer.

A hemostasis valve hub 212 (hereinafter “hub 212” for brevity) may be provided at the proximal end region 202 to provide access to the lumen 208 of the introducer sheath 200. The hub 212 may be configured for hemostasis by, for example, helping to prevent blood from leaking out of the introducer sheath 200 during use. For example, a medical device 230, such as a catheter or blood pump, may be inserted through the hub 212 and lumen 208 of the introducer sheath 200 and into the blood vessel V, and the hub 212 may maintain hemostasis between the medical device 230, the introducer sheath 200, and the external surroundings.

In some embodiments, the elongate shaft 206 of the introducer sheath 200 may include an actuatable fluid bypass mechanism 214. Generally, the fluid bypass mechanism 214 may be a movable door, flap, window, or the like 216 formed in the elongate shaft 206 of the introducer sheath 200. For example, the flap 216 may be articulatable along an edge thereof to selectively define an opening extending through the sidewall of the elongate shaft 206. In FIG. 3A, the flap 216 is in a closed configuration such that the flap 216 may be in line with the sidewall of the elongate shaft 206. In FIG. 3B, the flap 216 is deflected radially inwards to an open configuration to fluidly couple the lumen 208 of the introducer sheath 206 with the lumen of the vessel V. It is contemplated that the flap 216 may be positioned in the closed configuration during insertion and removal of the medical device 230 and opened while the medical device 230 remains in situ to allow for blood within the lumen 208 of the introducer sheath 200 to exit the lumen 208 and re-enter the vessel V. This may increase the amount of blood perfused past the introducer sheath 200 relative to a device free from a fluid bypass mechanism 214.

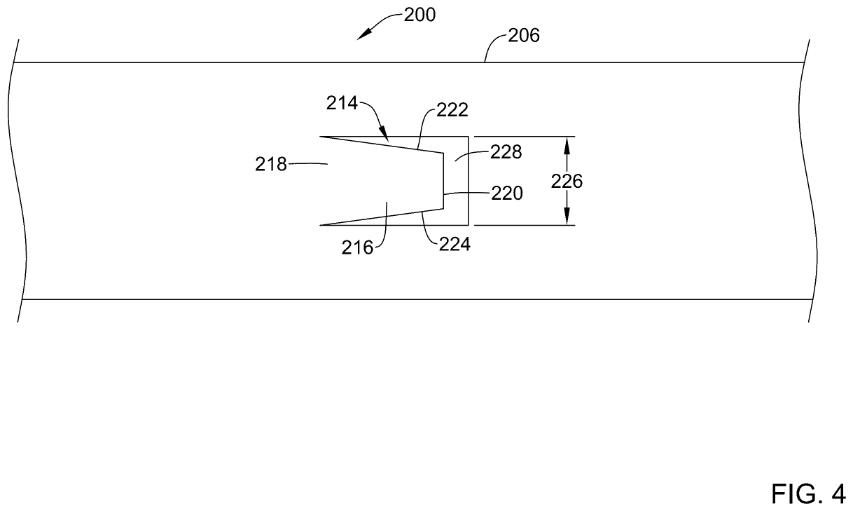

FIG. 4 illustrates a partial bottom view of the introducer sheath 200 adjacent to the fluid bypass mechanism 214 with the fluid bypass mechanism 214 is illustrated in the open configuration. The fluid bypass mechanism 214 may be formed as a single monolithic structure with the elongate shaft 206. For example, the flap 216 of the fluid bypass mechanism 214 may be formed by cutting through a sidewall of the elongate shaft 206 to form a flap 216 extending from a first fixed end 218 coupled to or monolithic with the elongate shaft 206 to a second free end 220 free from fixation to the elongate shaft 206 of the introducer sheath 200. The flap 216 may include a first lateral side 222 extending from the first end 218 to the second end 220 and a second lateral side 224 extending from the first end 218 to the second end 220. The first and second lateral sides 222, 224 may be free from fixation to the elongate shaft 206 of the introducer sheath 200 such that the flap 216 is pivotable around or at the first fixed end 218 thereof. The flap 216 may have a length 238 extending from the first fixed end 218 to the second free end 220. The length 238 of the flap 216 may be variable. In some embodiments, the length 238 may be selected such that the second free end 220 of the flap 216 contacts an outer surface of the medical device 230 when the fluid bypass mechanism 214 is in the open configuration. In other embodiments, the length 238 may be selected such that the second free end 220 is spaced a distance from or is free from contact with the outer surface of the medical device 230 when the fluid bypass mechanism 214 is in the open configuration.

The first lateral side 222 may be laterally spaced from the second lateral side 224 by an arc length 226. It is contemplated that the flap 216 may have a curvature similar to a curvature of the elongate shaft 206, thus the arc length 226 of the flap 216 may be greater than a linear distance between the first lateral side 222 and the second lateral side 224. It is contemplated that the arc length 226 between the first lateral side 222 and the second lateral side 224 of the flap 216 may be varied. In some cases, the arc length 226 may be about one-third of the circumference the elongate shaft 206 (e.g., may extend about 120° around the circumference of the elongate shaft 206). In other embodiments, the arc length 226 may be greater than one-third of the circumference the elongate shaft 206. In yet other embodiments, the arc length 226 may be less than one-third of the circumference of the elongate shaft 206.

The fluid bypass mechanism 214 may be positioned distal to the hub 212 and spaced therefrom such that when the introducer sheath 200 is disposed within the vessel V, the fluid bypass mechanism 214 is fully disposed within the vessel V. In some cases, the fluid bypass mechanism 214 may be spaced in the range of about 3 centimeters (cm) to about 7 cm from the hub 212. It is contemplated that the position of the fluid bypass mechanism 214 may vary based on the target anatomy and/or patient demographics and can be less than 3 cm or more than 7 cm from the hub 212.

In some embodiments, the fluid bypass mechanism 214 may be manually actuated or moved between the closed configuration (FIG. 3A) and the open configuration (FIG. 3B). For example, a distal end of a pull wire 232 or other actuation mechanism may be coupled to a radially inward portion of the flap 216. The pull wire 232 may extend proximally from the fluid bypass mechanism 214 to the hub 212. A proximal end of the pull wire 232 may be coupled or secured to an actuation mechanism 234. Some illustrative actuation mechanisms 234 may include, but are not limited to, toggles, thumb switches, roller wheels, or the like. The actuation mechanism 234 may be movable between a first closed configuration and a second open configuration. In one illustrative example, the actuation mechanism 234 may be distally advanced to close the fluid bypass mechanism 214 and proximally retracted to open the fluid bypass mechanism 214. However, in other examples, the actuation mechanism 234 may be proximally retracted to close the fluid bypass mechanism 214 and distally advanced to open the fluid bypass mechanism 214. The hub 212 may include visual indicia configured to indicate whether the fluid bypass mechanism 214 is in an open or closed configuration. In the illustrated embodiment, the hub 212 includes the letter “C” to indicate the fluid bypass mechanism 214 is in a closed configuration and the letter “O” to indicate the fluid bypass mechanism 214 is in an open configuration. However, other visual indicia may be used as desired, including, but not limited to, colors, shapes, patterns, letters, words, etc.

The distal end of the pull wire 232 may be fixedly or releasably coupled to the flap 216. As the pull wire 232 is actuated (via the actuation mechanism 234) from the closed configuration to the open configuration, the pull wire 232 may exert a proximal force on the flap 216 which draws the flap 216 radially inwards. When the pull wire 232 is actuated (via the actuation mechanism 234) from the open configuration to the closed configuration, the pull wire 232 may exert a distally pushing force on the flap 216 which moves the flap 216 radially outwards to align with the remaining elongate shaft 206. In some embodiments, the flap 216 may be formed from a shape memory material and may be heat set in the closed configuration. When the flap 216 is heat set in the closed configuration, a pushing force may not be required to move and/or maintain the flap 216 in the closed configuration, as the flap 216 may assume the closed configuration when not subjected to an applied, external force. In the closed configuration, the flap 216 may extend generally parallel to the longitudinal axis of the introducer sheath 200. Removal of the pulling force on the flap 216 may allow the flap 216 to assume its preset or unbiased (e.g., closed) configuration. The reverse configuration in which the flap 216 is heat set to the open configuration is also contemplated. The fluid bypass mechanism 214 may be closed during insertion and/or removal of the medical device 230 and opened while the medical device 230 remains in the body. As shown at arrows 236 in FIG. 3B, blood may flow through the lumen 208 of the introducer sheath 200, out the opening 228 created in the elongate shaft 206 of the introducer sheath 200 by opening or actuating the fluid bypass mechanism 214, and into the lumen of the vessel V.

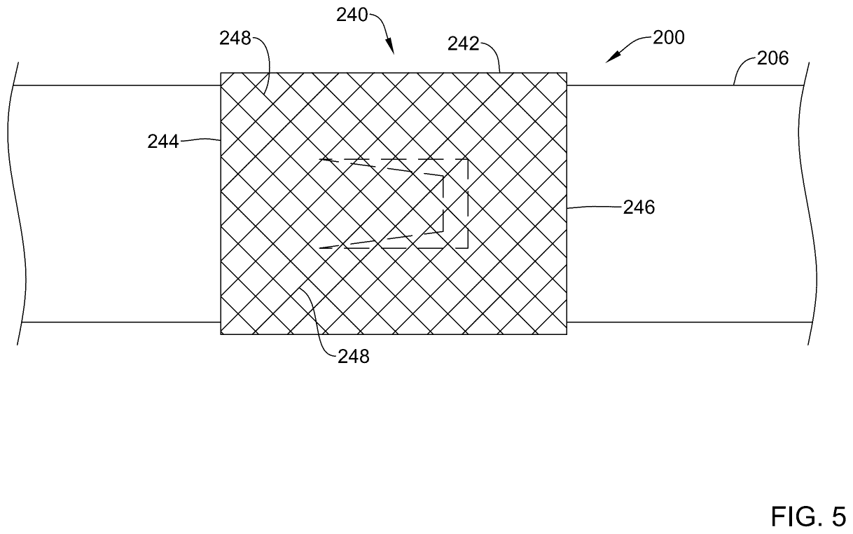

In some embodiments, a mesh or screen 240 may be positioned over the elongate shaft 206 adjacent to the fluid bypass mechanism 214. FIG. 5 illustrates a partial bottom view of the introducer sheath 200 adjacent to the fluid bypass mechanism 214 in an open configuration and having a mesh or screen 240 disposed over an outer surface of the elongate shaft 206 of the introducer sheath 200. In some instances, the mesh 240 may be formed from an elongated tubular member 242. While the mesh 240 is described as generally tubular, it is contemplated that the mesh 240 may take any cross-sectional shape desired. The mesh 240 may have a first, or proximal, end 244, a second, or distal, end 246, and an intermediate region disposed between the first end 244 and the second end 246. The mesh 240 may include a lumen extending from a first opening adjacent the first end 244 to a second opening adjacent to the second end 246 to allow the mesh 240 to surround the outer surface of the elongate shaft 206 of the introducer sheath 200.

The tubular member 242 of the mesh 240 may have a scaffold structure, fabricated from one or more, or a plurality of interwoven filaments or struts 248. The scaffold structure may extend from the first end 244 to the second end 246 of the mesh 240. For example, the scaffold structure, and thus the filament(s) thereof, may extend continuously from the first end 244 to the second end 246 of the mesh 240. In some embodiments, the mesh 240 may be formed with one filament interwoven with itself (e.g., knitted) to form the scaffold structure. In other embodiments, the mesh 240 may be formed with several interwoven filaments (e.g., braided) to form the scaffold structure. Thus, in such instances one or more of the filament(s) forming the scaffold structure may extend continuously from the first end 244 to the second end 246 of the mesh 240. In still another embodiment, the mesh 240 may include a laser cut tubular member to form the scaffold structure. A laser cut tubular member may have an open and/or closed cell geometry including one or more interconnected struts formed as a monolithic structure from the tubular member. In such instances, the laser cut tubular member forming the scaffold structure may extend continuously from the first end 244 to the second end 246 of the mesh 240.

It is contemplated that the scaffold structure, e.g., the filaments and/or struts, of the mesh 240 can be made from a number of different materials such as, but not limited to, metals, metal alloys, shape memory alloys, and/or polymers, as desired, enabling the mesh 240 to be positioned over the elongate shaft 206 of the introducer sheath 200. The mesh 240 may be fixedly or removably secured to the outer surface of the elongate shaft 206 using a number of different techniques including, but not limited to, heat shrinking, adhesives, friction fit, thermal welding, welding, soldering, brazing, or the like.

The mesh 240 may have a length that is at least as long as the length 238 of the flap 216, such that the mesh is disposed over an entirety of the length 238 of the flap 216. Further, while the mesh 240 is described as tubular, in some cases, the mesh 240 may extend around less than an entirety of the outer circumference of the introducer sheath 200. In such an instance, the mesh 240 may have an arc length that is at least as long as the arc length 226 of the flap 216. The mesh 240 may be disposed over an entirety of the opening 228 created in the elongate shaft 206 of the introducer sheath 200 when the fluid bypass mechanism 214 is in the open configuration. This may prevent particulates, debris, etc. that may have entered the lumen 208 of the introducer sheath 200 from re-entering the vessel V.

In some embodiments, the mesh 240 may include a drug eluting coating or therapeutic coating disposed on one or more surfaces thereof. In some cases, the drug coating or coating composition may include a direct oral anticoagulant (DOAC) on a surface of the mesh 240. DOACs bind directly to specific clotting factors. Examples of DOACs include apixaban, rivaroxaban, edoxaban, betrixaban, and argatroban, which directly bind to factor Xa, and dabigatran, which directly binds to factor IIa. However, other beneficial agents may include anti-thrombotic agents, anti-proliferative agents, anti-inflammatory agents, anti-migratory agents, agents affecting extracellular matrix production and organization, antineoplastic agents, anti-mitotic agents, anesthetic agents, anti-coagulants, vascular cell growth promoters, vascular cell growth inhibitors, cholesterol-lowering agents, vasodilating agents, and agents that interfere with endogenous vasoactive mechanisms.

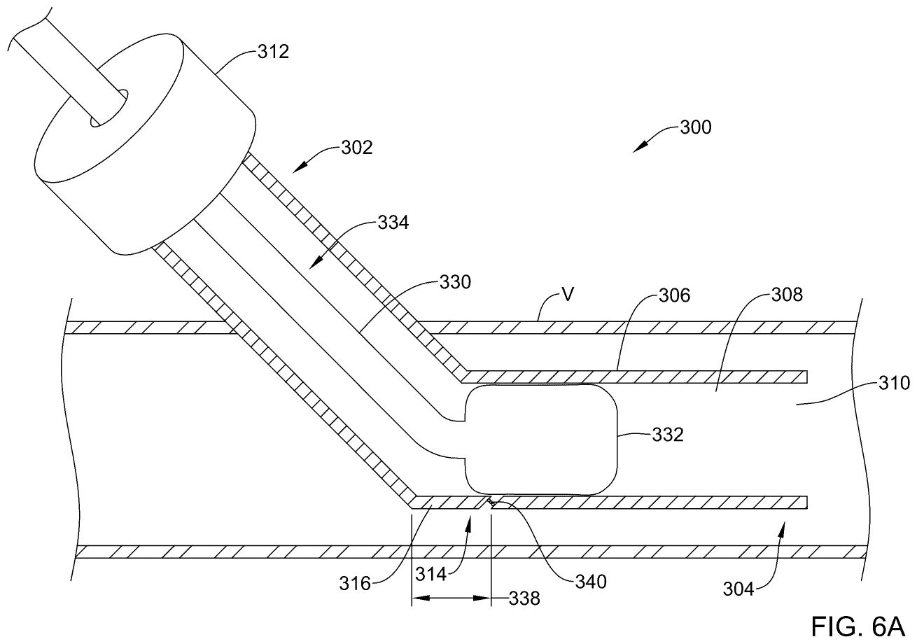

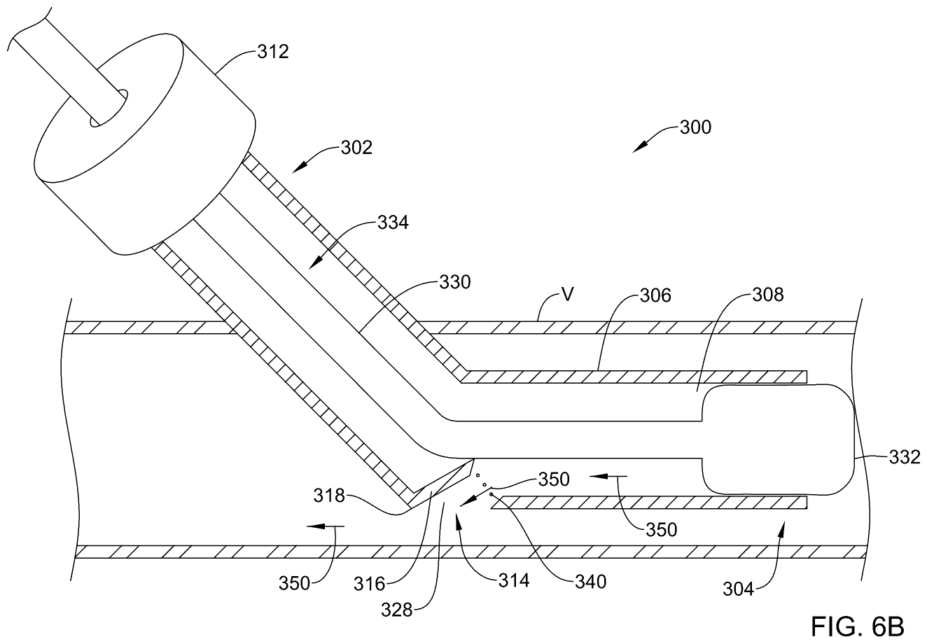

FIG. 6A is a partial cross-sectional view of another illustrative introducer sheath 300 disposed within a vessel V and in a first, closed, configuration and FIG. 6B is a partial cross-sectional view of the illustrative introducer sheath 300 disposed within a vessel V and in a second, open, configuration. The introducer sheath 300 includes a proximal end region 302 proximate a proximal end of the introducer sheath 300 and a distal end region 304 proximate a distal end of the introducer sheath 300 that is opposite the proximal end region 302. While FIG. 6B shows the distal end region 304 of the introducer sheath 300 proximal to a distal end region 332 of the medical device 330, the distal end region 304 of the introducer sheath 300 may extend distal to a distal end region 332 of the medical device 330 or may be adjacent to or near the distal end region 332 of the medical device 330. An elongate shaft 306 of the introducer sheath 300 extends between the proximal end region 302 and the distal end region 304, and the elongate shaft 306 defines a lumen 308 of the introducer sheath 300. The introducer sheath 300 includes a proximal opening (not shown) adjacent to the proximal end region 302 and a distal opening 310 adjacent to the distal end region 304, with the lumen 308 extending from the proximal opening to the distal opening 310. The introducer sheath 300, or components thereof, may be formed by various materials, such as polymeric and/or metallic materials. In some instances, the introducer sheath 300, such as the elongate shaft of the introducer sheath 300, may include an additional surface coating, such as but not limited to, silicone, polyethylene terephthalate (PET), or other applicable polymer.

A hemostasis valve hub 312 (hereinafter “hub 312” for brevity) may be provided at the proximal end region 302 to provide access to the lumen 308 of the introducer sheath 300. The hub 312 may be configured for hemostasis by, for example, helping to prevent blood from leaking out of the introducer sheath 300 during use. For example, a medical device 330, such as a catheter or blood pump, may be inserted through the hub 312 and lumen 308 of the introducer sheath 300 and into the blood vessel V, and the hub 312 may maintain hemostasis between the medical device 330, the introducer sheath 300, and the external surroundings.

In some embodiments, the elongate shaft 306 of the introducer sheath 300 may include an actuatable fluid bypass mechanism 314. Generally, the fluid bypass mechanism 314 may be a movable door, flap, window, or the like 316 formed in the elongate shaft 306 of the introducer sheath 300. For example, the flap 316 may be articulatable along an edge thereof to selectively define an opening extending through the sidewall of the elongate shaft 306. In FIG. 6A, the flap 316 is in a closed configuration such that the flap 316 may be in line with or biased towards the sidewall of the elongate shaft 306. In FIG. 6B, the flap 316 is disposed radially inwards to an open configuration to fluidly couple the lumen 308 of the introducer sheath 306 with the lumen of the vessel. It is contemplated that the flap 316 may be biased into the closed configuration during insertion and removal of the medical device 330 and opened while the medical device 330 remains in situ to allow for blood within the lumen 308 of the introducer sheath 300 to exit the lumen 308 and re-enter the vessel. This may increase the amount of blood perfused past the introducer sheath 300 relative to a device free from a fluid bypass mechanism 314. It is contemplated that the fluid bypass mechanism 314 may be configured to automatically return to the open configuration in the absence of a biasing force moving said fluid bypass mechanism 314 into the closed configuration. For example, when the introducer sheath 300 is introduced into the vessel V, a dilator (not explicitly shown) may be inserted into the lumen 310 of the introducer sheath 300 to maintain the flap 316 in the closed configuration. The flap 316 may remain in the closed configuration until the dilator is removed. Thus, the dilator may deflect the flap 316 away from its open configuration to the closed configuration. Additionally, the medical device 330 may have an enlarged distal end region 332 having a cross-sectional dimension greater than a proximal end region 334 thereof. As the enlarged distal end region 332 passes the fluid bypass mechanism 314, the flap 316 may be pushed towards the closed configuration. Alternatively, or additionally, a dilator may be inserted into the lumen 308 of the introducer sheath 300 during insertion and/or retraction of the medical device 330 to hold the fluid bypass mechanism 314 closed to prevent the medical device 330 from catching on the fluid bypass mechanism 314. In the absence of a dilator, the fluid bypass mechanism 314 may remain open during the removal of the medical device 330 or at least until the distal end region 332 of the medical device 330 is disposed adjacent to the fluid bypass mechanism 314. As shown at arrows 350 in FIG. 6B, blood may flow through the lumen 308 of the introducer sheath 300, out an opening 328 created in the elongate shaft 306 of the introducer sheath 300 by opening or actuating the fluid bypass mechanism 314, and into the lumen of the vessel V.

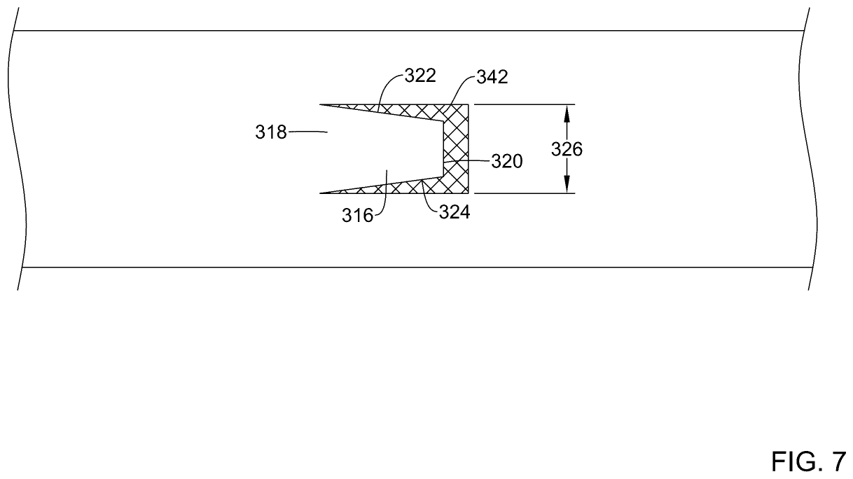

Referring additionally to FIG. 7 which illustrates a partial bottom view of the introducer sheath 300 adjacent to the fluid bypass mechanism 314, the fluid bypass mechanism 314 may be formed at least in part from a portion of the elongate shaft 306. For example, the flap 316 of the fluid bypass mechanism 314 may be formed by cutting through a sidewall of the elongate shaft 306 to form a flap 316 extending from a first fixed end 318 coupled to or monolithic with the elongate shaft 306 to a second end 320. The flap 316 include a first lateral side 322 extending from the first end 318 to the second end 320 and a second lateral side 324 extending from the first end 318 to the second end 320. The flap 316 may have a length 338 extending from the first fixed end 318 to the second end 320. The length 338 of the flap 316 may be variable. In some embodiments, the length 338 may be selected such that the second end 320 of the flap 316 contacts an outer surface of the medical device 330 when the flap 316 is in an open configuration. In other embodiments, the length 338 may be selected such that the second end 320 is spaced a distance from or is free from contact with the outer surface of the medical device 330 when the flap 316 is in an open configuration.

The first lateral side 322 may be laterally spaced from the second lateral side 324 by an arc length 326. It is contemplated that the flap 316 may have a curvature similar to a curvature of the elongate shaft 306, thus the arc length 326 of the flap 316 may be greater than a linear distance between the first lateral side 322 and the second lateral side 324. It is contemplated that the arc length 326 between the first lateral side 322 and the second lateral side 324 of the flap 316 may be varied. In some cases, the arc length 326 may be about one-third of the circumference the elongate shaft 306 (e.g., may extend about 120° around the circumference of the elongate shaft 306). In other embodiments, the arc length 326 may be greater than one-third of the circumference the elongate shaft 306. In yet other embodiments, the arc length 326 may be less than one-third of the circumference of the elongate shaft 306.

The flap 316 may be movably secured to the elongate shaft 306 at the second end 320, first lateral side 322 and/or second lateral side 324 via a biasing member 340. The biasing member 340 may be a shape memory material and may be heat set to hold the fluid bypass mechanism 314 in the open configuration. For example, the biasing member 340 may be heat set and biased at a desired pre-determined angle relative to a longitudinal axis of the introducer sheath 300. The angle may be between 5 degrees and 90 degrees. In some examples, the angle may be between 10 degrees and 45 degrees. In some examples, the biasing member 340 may extend between each of the second end 320, the first lateral side 322, and the second lateral side 324 and the elongate shaft 306. However, this is not required. In some embodiments, the biasing member 340 may extend between only one of the second end 320, the first lateral side 322, or the second lateral side 324 and the elongate shaft 306. In other examples, the biasing member 340 may extend between any two of the second end 320, the first lateral side 322, and the second lateral side 324 and the elongate shaft 306.

The biasing member 340 may have a scaffold structure, fabricated from one or more, or a plurality of interwoven filaments or struts 342. The scaffold structure may extend along portions or an entirety of a perimeter of the flap 316. It is contemplated that in some cases the biasing member 340 may extend along more than one discrete portion of the flap 316. For example, discrete portions of the biasing member 340 may be separated by portions free from the biasing member 340. In some embodiments, the biasing member 340 may be formed with one filament interwoven with itself (e.g., knitted) to form the scaffold structure. In other embodiments, the biasing member 340 may be formed with several interwoven filaments (e.g., braided) to form the scaffold structure. Thus, in such instances one or more of the filament(s) forming the scaffold structure may extend continuously from a first end to a second end of the biasing member 340. In still another embodiment, the biasing member 340 may include a laser cut member to form the scaffold structure. A laser cut member may have an open and/or closed cell geometry including one or more interconnected struts formed as a monolithic structure. The scaffold structure may prevent particulates, debris, etc. that may have entered the lumen 308 of the introducer sheath 300 from re-entering the vessel V.

It is contemplated that the scaffold structure, e.g., the filaments and/or struts, of the biasing member 340 can be made from a number of different materials such as, but not limited to, metals, metal alloys, shape memory alloys, and/or polymers, as desired, enabling the biasing member 340 to be secured to the elongate shaft 306 of the introducer sheath 300 and the flap 316 of the fluid bypass mechanism 314. The biasing member 340 may be fixedly or removably secured to the elongate shaft 306 using a number of different techniques including, but not limited to, heat shrinking, adhesives, friction fit, thermal welding, welding, soldering, brazing, or the like. Alternatively, the biasing member 340 may be formed as a monolithic structure with the elongate shaft 306.

In some embodiments, the biasing member 340 may include a drug eluting coating or therapeutic coating disposed on one or more surfaces thereof. In some cases, the drug coating or coating composition may include a direct oral anticoagulant (DOAC) on a surface of the biasing member 340. However, other beneficial agents may include anti-thrombotic agents, anti-proliferative agents, anti-inflammatory agents, anti-migratory agents, agents affecting extracellular matrix production and organization, antineoplastic agents, anti-mitotic agents, anesthetic agents, anti-coagulants, vascular cell growth promoters, vascular cell growth inhibitors, cholesterol-lowering agents, vasodilating agents, and agents that interfere with endogenous vasoactive mechanisms.

The fluid bypass mechanism 314 may be positioned distal to the hub 312 and spaced therefrom such that when the introducer sheath 300 is disposed within the vessel V, the fluid bypass mechanism 314 is fully disposed within the vessel V. In some cases, the fluid bypass mechanism 314 may be spaced in the range of about 3 centimeters (cm) to about 7 cm from the hub 312. It is contemplated that the position of the fluid bypass mechanism 314 may vary based on the target anatomy and/or patient demographics and can be less than 3 cm or more than 7 cm from the hub 312.

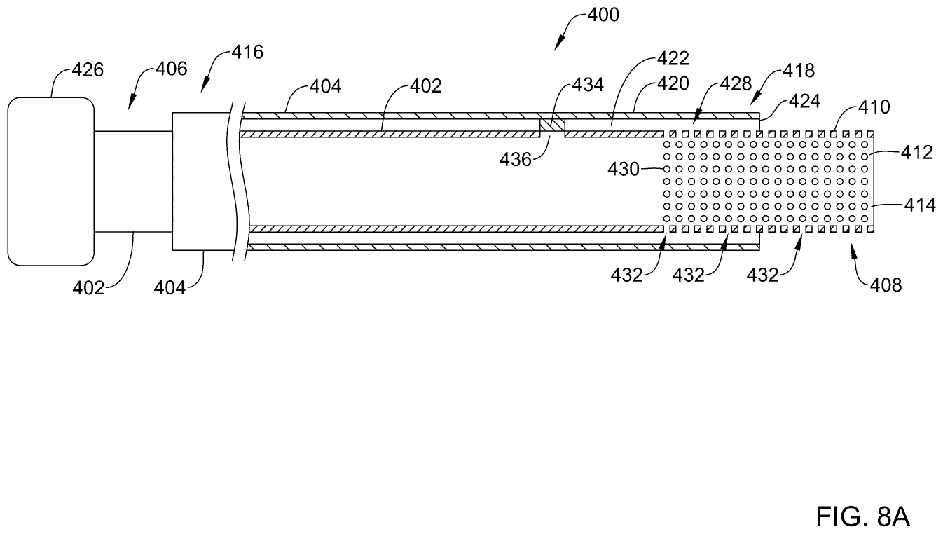

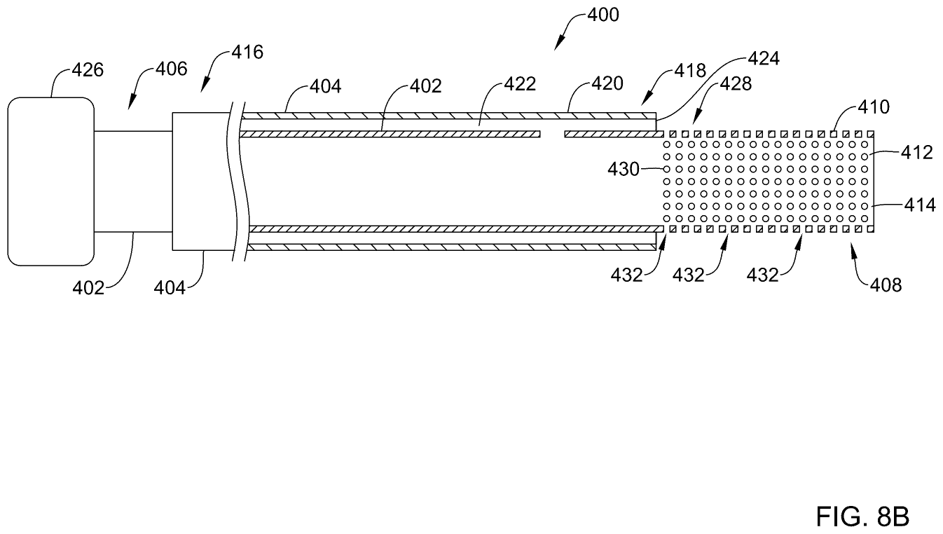

FIG. 8A is a partial cross-sectional view of another illustrative introducer sheath 400 in a first configuration and FIG. 8B is a partial cross-sectional view of the illustrative introducer sheath 400 in a second configuration. The introducer sheath 400 may include an inner tubular member 402 and an outer tubular member 404. As will be described in more detail herein, the outer tubular member 404 may be configured to be axially and/or rotationally displaced relative to the inner tubular member 402. The introducer sheath 400, or components thereof, may be formed by various materials, such as polymeric and/or metallic materials. In some instances, the introducer sheath 400, such as the elongate shaft(s) of the introducer sheath 400, may include an additional surface coating, such as but not limited to, silicone, polyethylene terephthalate (PET), or other applicable polymer.

The inner tubular member 402 extends from a proximal end region 406 proximate a proximal end of the introducer sheath 400 to a distal end region 408 proximate a distal end of the introducer sheath 400 that is opposite the proximal end region 406. An elongate shaft 410 of the inner tubular member 402 extends between the proximal end region 406 and the distal end region 408, and the elongate shaft 410 defines a lumen 412 of the inner tubular member 402. The inner tubular member 402 includes a proximal opening (not shown) adjacent the proximal end region 406 and a distal opening 414 adjacent the distal end region 408, with the lumen 412 extending from the proximal opening to the distal opening 414. In some embodiments, the distal end region 408 of the inner tubular member 402 may be proximal to a distal end region of a medical device (not explicitly shown). In other embodiments, the distal end region 408 of the inner tubular member 402 may extend distal to a distal end region of the medical device or may be adjacent to or near the distal end region of the medical device.

The outer tubular member 404 extends from a proximal end region 416 proximate a proximal end of the introducer sheath 400 to a distal end region 418 proximate a distal end of the introducer sheath 400 that is opposite the proximal end region 416. An elongate shaft 420 of the outer tubular member 404 extends between the proximal end region 416 and the distal end region 418, and the elongate shaft 420 defines a lumen 422 of the outer tubular member 404. The outer tubular member 404 includes a proximal opening (not shown) adjacent the proximal end region 416 and a distal opening 424 adjacent the distal end region 418, with the lumen 422 extending from the proximal opening to the distal opening 414. In some embodiments, the outer tubular member 404 may have a length (e.g., distance from the proximal end region 416 to the distal end region 418) that is less than a length of the inner tubular member 402. However, this is not required.

A hemostasis valve hub 426 (hereinafter “hub 426” for brevity) may be provided at the proximal end region 406 of the inner tubular member 402 to provide access to the lumen 412 of the inner tubular member 402. For example, a medical device may be advanced through the hub 426 and the lumen 412 of the inner tubular member 402 to the target location. The hub 426 may be configured for hemostasis by, for example, helping to prevent blood from leaking out of the introducer sheath 400 during use. For example, a medical device, such as a catheter or blood pump, may be inserted through the hub 426 and lumen 412 of the inner tubular member and into a blood vessel, and the hub 426 may maintain hemostasis between the medical device, the introducer sheath 400, and the external surroundings.

In some embodiments, the elongate shaft 410 of the inner tubular member 402 may include a fluid bypass mechanism 428. Generally, the fluid bypass mechanism 428 may allow a flow of blood or fluid to exit the lumen 412 of the inner tubular member 402 and enter the vessel lumen. The fluid bypass mechanism 428 may include a plurality of apertures, openings, slots, or the like 430 extending through a thickness of the sidewall of the elongate shaft 410 of the inner tubular member 402. For example, one or more apertures 430 may extend from an inner surface of the inner tubular member 402 to an outer surface of the inner tubular member 402 to fluidly couple the lumen 412 of the inner tubular member 402 with the exterior thereof. The apertures 430 may be arranged in any pattern desired. In the illustrated embodiment, the apertures 430 are arranged in circumferentially extending arrays 432. The arrays 432 may include a same number of apertures 430 or differing numbers of aperture 430, as desired. The apertures 430 may be spaced around an entirety of the circumference of the inner tubular member 402 or may be spaced around less than entirety of the circumference of the inner tubular member 402. In other examples, the apertures 430 may be arranged in a helical pattern. In yet other examples, the apertures 430 may be arranged in a non-uniform or asymmetric arrangement. It is further contemplated that the apertures 430 may have circular or non-circular cross-sectional shapes as desired. Some illustrative cross-sectional shapes may include, but are not limited to, square, rectangular, oblong, polygonal, stadium shaped, triangular, or the like. The apertures 430 may be formed in the inner tubular member 402 during manufacture of the inner tubular member 402 (e.g., molded therein) or may be formed after manufacture of the inner tubular member 402 (e.g., cut, drilled, laser cut, etc.).

In the illustrated embodiment, the apertures 430 extend proximally from the distal end region 408 of the inner tubular member 402. However, this is not required. In some examples, the apertures 430 may not extend entirely to the distal end region 408. It is further contemplated that the apertures 430 may be provided along more than one section of the elongate shaft 410. For example, the inner tubular member 402 may include more than one fluid bypass mechanism 428 spaced along a length thereof. The apertures 430 may be along an entirety of the length of the inner tubular member 402 or a portion thereof, as desired.

The outer tubular member 404 may be selectively disposed over the fluid bypass mechanism 428 to selectively fluidly couple the lumen 412 of the inner tubular member 402 with the body lumen. For example, during insertion of the introducer sheath 400, the outer tubular member 404 may be distally advanced (as shown in FIG. 8A) to surround or cover all or portions of the fluid bypass mechanism 428. In some examples, the outer tubular member 404 may have a length such that the outer tubular member 404 extends distally beyond a distal end region 408 of the inner tubular member 402. When the medical device is in position, the outer tubular member 404 be proximally retracted (as shown in FIG. 8B) to expose all or portions of the fluid bypass mechanism 428 and allow fluid within the lumen 412 of the inner tubular member 402 to exit via the apertures 430.

In some embodiments, the inner tubular member 402 and/or the outer tubular member 404 may include features to prevent accidental actuation of the outer tubular member 404 and/or to movably secure the outer tubular member 404 to the inner tubular member 402. In one illustrative example, the outer tubular member 404 may include a radially inwardly extending protrusion 434. The radially inwardly extending protrusion 434 may be configured to engage a mating slot or recess 436 in the inner tubular member 402. It is contemplated that the mating slot or recess 436 may be bent or curved such that the outer tubular member 404 must first be rotated prior to proximal (or distal) movement thereof. For example, the slot or recess 436 may include a circumferentially extending portion at a proximal and/or distal end thereof and a longitudinally extending portion therebetween. Other configurations may be used as desired. Once the outer tubular member 404 has been rotated, the outer tubular member 404 may be proximally retracted or distally advanced within a longitudinally extending portion of the slot or recess 436. Once the protrusion 434 engages an end of the slot 436, the outer tubular member 404 may again be rotated into a circumferentially extending portion of the slot 436 to prevent further longitudinal movement of the outer tubular member 404. In some examples, the retraction of the outer tubular sheath 404 may be variable to adjust an amount of the fluid bypass mechanism 428 that is exposed. It is contemplated that in some examples, the outer tubular member 404 may be omitted such that no active actuation is required to expose the fluid bypass mechanism 428.

In some embodiments, a mesh or screen similar in form and function to the mesh 240 described herein may be positioned over the elongate shaft 410 of the inner tubular member adjacent to the fluid bypass mechanism 428. In some instances, the mesh may be formed from an elongated tubular member. While the mesh is described as generally tubular, it is contemplated that the mesh may take any cross-sectional shape desired. The tubular member of the mesh may have a scaffold structure, fabricated from one or more, or a plurality of interwoven filaments or struts. In some embodiments, the mesh may be formed with one filament interwoven with itself (e.g., knitted) to form the scaffold structure. In other embodiments, the mesh may be formed with several interwoven filaments (e.g., braided) to form the scaffold structure. In still another embodiment, the mesh may include a laser cut tubular member to form the scaffold structure. A laser cut tubular member may have an open and/or closed cell geometry including one or more interconnected struts formed as a monolithic structure from the tubular member.

It is contemplated that the scaffold structure, e.g., the filaments and/or struts, of the mesh can be made from a number of different materials such as, but not limited to, metals, metal alloys, shape memory alloys, and/or polymers, as desired, enabling the mesh to be positioned over the elongate shaft 410 of the inner tubular member 410. The mesh may be fixedly or removably secured to the outer surface of the elongate shaft 410 using a number of different techniques including, but not limited to, heat shrinking, adhesives, friction fit, thermal welding, welding, soldering, brazing, or the like.

The mesh may have a length that is at least as long as the length of the fluid bypass mechanism 428, such that the mesh is disposed over an entirety of the length of the fluid bypass mechanism 428. Further, while the mesh is described as tubular, in some cases, the mesh may extend about less than an entirety of the outer circumference of the inner tubular member 402. The mesh 240 may be disposed over an entirety of the apertures 430. This may prevent particulates, debris, etc. that may have entered the lumen 412 of the inner tubular member 402 from re-entering the vessel V.

In some embodiments, the mesh may include a drug eluting coating or therapeutic coating disposed on one or more surfaces thereof. In some cases, the drug coating or coating composition may include a direct oral anticoagulant (DOAC) on a surface of the mesh. DOACs bind directly to specific clotting factors. Examples of DOACs include apixaban, rivaroxaban, edoxaban, betrixaban, and argatroban, which directly bind to factor Xa, and dabigatran, which directly binds to factor IIa. However, other beneficial agents may include anti-thrombotic agents, anti-proliferative agents, anti-inflammatory agents, anti-migratory agents, agents affecting extracellular matrix production and organization, antineoplastic agents, anti-mitotic agents, anesthetic agents, anti-coagulants, vascular cell growth promoters, vascular cell growth inhibitors, cholesterol-lowering agents, vasodilating agents, and agents that interfere with endogenous vasoactive mechanisms.

The fluid bypass mechanism 428 may be positioned distal to the hub 426 and spaced therefrom such that when the introducer sheath 400 is disposed within the vessel V, the fluid bypass mechanism 428 is fully disposed within the vessel V. In some cases, the fluid bypass mechanism 428 may be spaced in the range of about 3 centimeters (cm) to about 7 cm from the hub 426. It is contemplated that the position of the fluid bypass mechanism 428 may vary based on the target anatomy and/or patient demographics and can be less than 3 cm or more than 7 cm from the hub 426. In some examples, the fluid bypass mechanism 428 may be positioned at or adjacent to the distal end region 408 of the inner tubular member 402. In other examples, one or more fluid bypass mechanisms 428 may be positioned at various longitudinal positions along the length of the inner tubular member 402.

More specific drugs or therapeutic agents that may be used with the drug eluting coatigs include apixaban, rivaroxaban, edoxaban, dabigatran, betrixaban, argatroban, paclitaxel, rapamycin, sirolimus, everolimus, tacrolimus, heparin, diclofenac, aspirin, Epo D, dexamethasone, estradiol, halofuginone, cilostazol, geldanamycin, ABT-578 (Abbott Laboratories), trapidil, liprostin, actinomycin D, Resten-NG, Ap-17,abciximab, clopidogrel, Ridogrel, beta-blockers, bARKct inhibitors, phospholamban inhibitors, and SERCA 2 gene/protein, resiquimod, imiquimod (as well as other imidazoquinoline immune response modifiers), human apolipoproteins (e.g., AI, AII, AIII, AIV, AV, etc.), vascular endothelial growth factors (e.g., VEGF-2), as well as derivatives of the forgoing, among many others.

In some embodiments, the drug may be a macrolide immunosuppressive (limus) drug. In some embodiments, the macrolide immunosuppressive drug is rapamycin, biolimus (biolimus A9), 40-O-(2-Hydroxyethyl)rapamycin (everolimus), 40-O-Benzyl-rapamycin, 40-O-(4′-Hydroxymethyl)benzyl-rapamycin, 40-O-[4′-(1,2-Dihydroxyethyl)]benzyl-rapamycin, 40-O-Allyl-rapamycin, 40-O-[3′-(2,2-Dimethyl-1,3-dioxolan-4(S)-yl)-prop-2′-en-1′-yl]-rapamycin, (2′:E,4′S)-40-O-(4′,5′-Dihydroxypent-2′-en-1′-yl)-rapamycin, 40-O-(2-Hydroxy)ethoxycar-bonylmethyl-rapamycin, 40-O-(3-Hydroxy)propyl-rapamycin, 40-O-(6-Hydroxy)hexyl-rapamycin, 40-O-[2-(2-Hydroxy)ethoxy]ethyl-rapamycin, 40-O-[(3S)-2,2-Dimethyldioxolan-3-yl]methyl-rapamycin, 40-O-[(2S)-2,3-Dihydroxyprop-1-yl]-rapamycin, 40-O-(2-Acetoxy)ethyl-rapamycin, 40-O-(2-Nicotinoyloxy) ethyl-rapamycin, 40-O-[2-(N-Morpholino)acetoxl]ethyl-rapamycin, 40-O-(2-N-Imidazolylacetoxy)ethyl-rapamycin, 40-O-[2-(N-Methyl-N′-piperazinyl)acetoxy]ethyl-rapamycin, 39-O-Desmethyl-39,40-O,O-ethylene-rapamycin, (26R)-26-Dihydro-40-O-(2-hydroxy)ethyl-rapamycin, 28-O-Methyl-rapamycin, 40-O-(2-Aminocthyl)-rapamycin, 40-O-(2-Acetaminocthyl)-rapamycin, 40-O-(2-Nicotinamidocthyl)-rapamycin, 40-O-(2-(N-Methyl-imidazo-2′-ylcarbethoxamido)ethyl)-rapamycin, 40-O-(2-Ethoxycarbonylaminoethyl)-rapamycin, 40-O-(2-Tolylsulfonamidoethyl)-rapamycin, 40-O-[2-(4′,5′-Dicarbocthoxy-1′,2′,3′-triazol-1′-yl)-ethyl]-rapamycin, 42-Epi-(tetrazolyl) rapamycin (tacrolimus), 42-[3-hydroxy-2-(hydroxymethyl)-2-methylpropanoate]rapamycin (temsirolimus), (42S)-42-Deoxy-42-(1H-tetrazol-1-yl)-rapamycin (zotarolimus), or isomer, racemate, diastereoisomer, prodrug, hydrate, ester, or analog thereof. Other drugs may include anti-inflammatory agents such as dexamethasone, prednisolone, corticosterone, budesonide, estrogen, sulfasalazine, mesalamine, and analogues thereof; antineoplastic/antiproliferative/anti-miotic agents such as paclitaxel, 5-fluorouracil, cisplatin, vinblastine, vincristine, epothilones, endostatin, angiostatin, thymidine kinase inhibitors, and analogues thereof; anesthetic agents such as lidocaine, bupivacaine, ropivacaine, and analogues thereof; anti-coagulants; and growth factors.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A hub and sheath assembly, comprising:
a valve hub including a lumen extending therethrough;
an elongate shaft extending from a proximal end region to a distal end region and including a lumen extending therebetween, the elongate shaft coupled to the valve hub; and
a fluid bypass mechanism extending through a sidewall of the elongate shaft;
wherein the fluid bypass mechanism is configured to selectively fluidly couple the lumen of the elongate shaft with an exterior thereof.

2. The hub and sheath assembly of claim 1, wherein the fluid bypass mechanism comprises a movable flap formed in the sidewall of the elongate shaft.

3. The hub and sheath assembly of claim 2, wherein the movable flap is configured to be deflected radially inwards.

4. The hub and sheath assembly of claim 1, further comprising a pull wire coupled to the fluid bypass mechanism.

5. The hub and sheath assembly of claim 4, wherein the pull wire extends from the fluid bypass mechanism to the valve hub.

6. The hub and sheath assembly of claim 4, further comprising an actuation mechanism coupled to a proximal end of the pull wire.

7. The hub and sheath assembly of claim 6, wherein actuation of the actuation mechanism is configured to move the fluid bypass mechanism between a generally closed configuration and an open configuration.

8. The hub and sheath assembly of claim 1, further comprising a mesh positioned over the elongate shaft adjacent to the fluid bypass mechanism.

9. A hub and sheath assembly, comprising:
a valve hub including a lumen extending therethrough;
an elongate shaft extending from a proximal end region to a distal end region and including a lumen extending therebetween, the elongate shaft coupled to the valve hub; and
a fluid bypass mechanism extending through a sidewall of the elongate shaft, the fluid bypass mechanism configured to move between a first closed configuration and a second open configuration;
wherein the fluid bypass mechanism is configured to selectively fluidly couple the lumen of the elongate shaft with an exterior thereof.

10. The hub and sheath assembly of claim 9, wherein the fluid bypass mechanism is biased to the open configuration.

11. The hub and sheath assembly of claim 9, further comprising a biasing mechanism extending between the elongate shaft and at least one side of the flap.

12. The hub and sheath assembly of claim 11, wherein the biasing mechanism is configured to bias the flap into a radially inwards open configuration.

13. The hub and sheath assembly of claim 11, wherein the biasing mechanism comprises a shape memory material.

14. The hub and sheath assembly of claim 11, wherein the biasing mechanism comprises a scaffold structure.

15. A hub and sheath assembly, comprising:
a valve hub including a lumen extending therethrough;
an elongate shaft extending from a proximal end region to a distal end region and including a lumen extending therebetween, the elongate shaft coupled to the valve hub; and
a fluid bypass mechanism extending through a sidewall of the elongate shaft, the fluid bypass mechanism comprising a plurality of apertures extending through the sidewall of the elongate shaft.

16. The hub and sheath assembly claim 15, further comprising an outer tubular member movably disposed over the elongate shaft.

17. The hub and sheath assembly of claim 16, wherein rotational and/or longitudinal actuation of the outer tubular member is configured to selectively expose the fluid bypass mechanism.

18. The hub and sheath assembly of claim 15, wherein the plurality of apertures are arranged in one or more circumferential arrays.

19. The hub and sheath assembly of claim 16, wherein the outer tubular member comprises a radially inwardly extending protrusion, the radially inwardly extending protrusion configured to engage a mating slot formed in an outer surface of the elongate member.

20. The hub and sheath assembly claim 16, wherein the fluid bypass mechanism is positioned in the range of about 3 to 10 centimeters distal to the valve hub.
